(19) United States
(12) Patent Application Publication
(10) Pub. No.: US 2012/0142925 A1
(43) Pub. Date: Jun. 7, 2012

(54) PROCESS FOR THE DEMETHYLATION OF OXYCODONE AND RELATED COMPOUNDS

(75) Inventors: Ole Heine Kvernenes, Oslo (NO); Anne Mette Nygard, Jar (NO); Audun Hegges, Oslo (NO); Harald Haivorsen, Oslo (NO)

(73) Assignee: ALPHARMA ApS, Copenhagen (DK)

(21) Appl. No.: 12/300,068

(22) PCT Filed: May 25, 2007
(86) PCT No.: PCT/EP2007/004675

§ 371 Date: Aug. 24, 2009

(30) Foreign Application Priority Data
May 25, 2006 (GB) 0610387.3

(51) Int. Cl. C07D 489/02; C07D 489/00

(52) U.S. Cl. 546/45; 546/46

(57) ABSTRACT

A process is provided for the N-demethylation of certain morphinans by reaction with α-chloroethyl chloroformate followed by hydrolysis of the resulting intermediate.

The invention described herein relates to an improved process of N-dealkylation of codeinone and morphinone derivatives.

The structures of codeinone and morphinone are as follows:

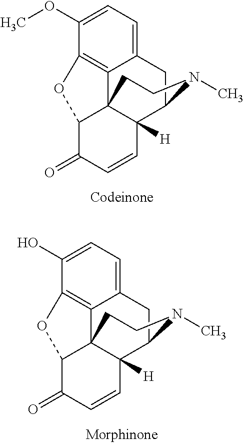

N-dealkylation of amines is a known synthetic reaction and can be carried out with common known reagents such as cyanogen bromide (BrCN) and various chloroformates. α-Chloroethylchloroformate (ACE-Cl) is known to N-dealkylate certain tertiary amines (J. Org. Chem., 1984, 49, 2081-2082).

N-Demethylation is often an important step in the chemical synthesis of codeinone-derivatives and morphinone-derivatives. However these derivatives contain a number of other functional groups which may react during the N-demethylation step. It has been shown that it is important when performing N-demethylation reactions to protect other functional groups present in the molecule.

The review contained in J. Org. Chem., Vol., 49, No 11, 1984, describes a reaction sequence wherein oxycodone is first acetylated to produce 14-acetyloxycodone and is then subsequently N-demethylated with ACE-Cl. Protection of the 14-OH is carried out despite the fact that other chloroformates, eg VOC-Cl had been used without protecting the 14-OH group (see U.S. Pat. No. 3,905,981 below). From this disclosure it was therefore understood that the 14-OH group should be protected when N-demethylating codeinone and morphinone derivatives. This understanding is further exemplified in the disclosures discussed below.

International Patent Application No WO 2005/107752 (see page 19) and U.S. Pat. No. 6,136,817 (see column 6) both disclose that codeinone derivatives with an alkoxy or an arylalkoxy group at the 14-position can be N-demethylated by reaction with chloroformates or cyanogen bromide.

U.S. patent application Ser. No. 10/519,388 (see paragraphs 126-132) teach that codeinone derivatives with an alkoxy, alkenyloxy, alkynyloxy, cycloalkylalkoxy at the 14-position can be N-demethylated by reaction with chloroformates or cyanogen bromide.

European Patent No 0 045 234 teaches that morphine, codeine, thebaine and N-alkyl 14-acyloxy morphinans can be dealkylated by using α-chloroethyl chloroformates (ACE-Cl).

European Patent No 0 164 290 discloses that the dealkylation of morphinan alkaloids with an ester group at the 14 position can be carried out by reaction with ethyl chloroformate followed by hydrolysis in a strong acid medium.

U.S. Pat. No. 3,905,981 describes the use of vinyl chloroformate (VOC) for N-dealkylating tertiary amines.

U.S. Pat. No. 4,472,253 discloses a N-demethylation reaction of codeine or 3-O-alkylmorphines with a cyanogen halide or haloformate. Neither codeine nor the 3-O-alkylmorphines have an OH group at the 14-position.

European Patent Application No 0 158 476 teaches a process for preparing noroxymorphone. The first step of the process is the reaction of morphine, having an H at the 14-position, with a haloformate ester. The noroxymorphone-ester undergoes a number of reaction steps before it is N-demethylated by hydrolysis.

A review contained in Organic Process Research and Development, 2004, 7, 279-282 describes an N-demethylation reaction by various chloroformates, including α-chloroethyl chloroformates of various δ-opioid antagonists. This document does not however disclose N-demethylation reactions of codeinone or morphinone derivatives which are the subject matter of this invention but instead the N-demethylation of compounds which are structurally very distinct to those which are the subject of this invention.

The above prior art indicates that a protecting group for the 14-hydroxy group of codienone and morphinone derivatives is desirable, for example to avoid by-product formation during the N-demethylation step. However it has now been surprisingly found that it is possible to perform the N-demethylation on codienone and morphinone compounds in which the 14-hydroxy group is not protected. This enables an efficient process with few steps to be achieved.

This therefore allows for a reduction in the total number of reaction steps needed as one protection step and one deprotection can be avoided. This therefore offers advantages in the commercial preparation of compounds formed via certain N-demethylated compounds.

The present invention provides a process for the preparation of a compound of formula (I), or a pharmaceutically acceptable salt thereof, for example the HCl salt

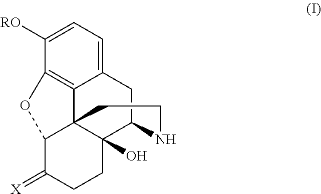

wherein X is CH₂ or O or X is a protected keto group and R is H, CH₃, O.CO.CH₃ or a silyl protecting group, or a salt thereof, which process comprises the reaction of a compound of the formula

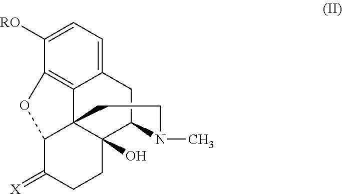

with α-chloroethyl chloroformate and hydrolysing the resulting intermediate, and optionally forming a pharmaceutical acceptable salt or free base as desired.

The intermediate will possess a carbamate in position 17 and generally also a carbonate at position 14. They both may be hydrolysed in a conventional manner.

In compounds of formula (I) and (II), R is preferably methyl.

In compounds of the formula (I) and (II), X is aptly O or a protected keto group. Suitable keto protecting group include ketals, for example, optionally linked diC₁₋₄ alkyl ketals. Particularly suitable protecting groups include those wherein X is a O(CH₂)ₙO group where n is 2 or 3, preferably 2.

In compounds of formula (I) and (II) X is most suitably O or OCH₂CH₂O and is preferably O.

Hence in a particularly preferred process according to the present invention, the compound of the formula (II) is oxycodone and the compound of the formula (I) is noroxycodone.

Therefore in a preferred aspect this invention provides a process for the preparation of the compound of the formula (III) or a pharmaceutically acceptable salt thereof, for example the HCl salt,

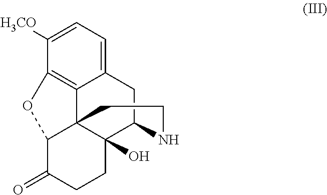

which process comprises the reaction of a compound of the formula (IV)

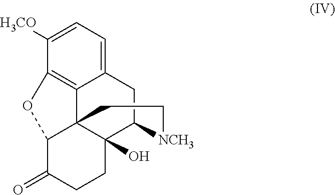

with α-chloroethylchloroformate and hydrolysing the resulting intermediate, and optionally forming a pharmaceutical acceptable salt or free base as desired.

Preferably the preceding processes do not involve isolation of the intermediate.

The N-demethylation reaction is most suitably performed in an aprotic solvent such as dichloromethane, dimethylformamide, acetonitrile, tetrahydrofuran, 1,2-dichloroethane, or the like. A favoured solvent is dichloromethane. Surprisingly a most preferable solvent is acetonitrile. It is surprising that acetonitrile is a preferable solvent because the review in Organic Process Research and Development, 2004, 8, 279-282 says that chlorinated solvents, such as dichloromethane, are required to make the reaction run to completion. The use of acetonitrile as opposed to dichloromethane is advantageous because it is less toxic, i.e. it is considered to be “greener”. In addition, the use of acetonitrile is advantageous as it is not necessary to remove the solvent upon completion of the reaction, and prior to precipitation of the hydrochloride salt. On the other hand, if the solvent used is dichloromethane, it is necessary to remove the solvent prior to precipitation of the hydrochloride salt. The use of acetonitrile as the solvent therefore means that one-less step is required in the overall process.

The N-demethylation reaction is most suitably carried out in the presence of a proton acceptor. Suitable proton acceptors include carbonates and bicarbonates, proton sponge, Hunig's base and the like. A particularly suitable proton acceptor is sodium carbonate, preferably anhydrous sodium carbonate. It has surprisingly been found that if the proton acceptor is added in more than one portion throughout the reaction, for example in two or three separate portions, higher yields can be obtained.

The use of C₁₋₄ alkanol, for example isopropanol, in the hydrolysis step is advantageous in order to help recover the product. After reaction the inorganic proton acceptor (base) may be filtered off and the desired product precipitated by adding wet isopropanol. Generally the product is obtained as its hydrochloride salt by precipitation at ambient temperature.

The N-demethylation reaction is suitably performed at a non-extreme temperature, for example, from ambient temperature up to the reflux temperature of the reaction mixture. Particularly suitably the reaction can be commenced and carried out at ambient temperature (for example 20-25° C.). Optionally the reaction can be progressed to a more elevated temperature, (for example 30-70° C., preferably 40-50° C. if desired).

The reaction may be performed under an inert atmosphere, for example nitrogen, in order to maintain a moisture free environment.

The solvent may be removed to yield the intermediate carbamate. This is then hydrolysed, for example by reaction with aqueous hydrochloric acid or with aqueous THF, for example at ambient temperature (for example 20-25° C.). It is preferable to hydrolyse by reaction with aqueous THF or aqueous isopropanol.

The reaction time may be significantly reduced with the addition of phase transfer catalysts, such as for example, tetrabutylammonium bromide (TBAB), hexadecyltrimethyl ammonium bromide, methyltrioctyl ammonium chloride, benzyltributyl ammonium chloride and tetrabutyl ammonium bisulfate. A preferable phase transfer catalyst is tetrabutylammonium bromide (TBAB).

The free base of the compound of formula (I) may be obtained by neutralisation of a salt of a compound of formula (I). A pharmaceutically acceptable salt of formula (I) may be obtained by mixing the free base or a salt of a compound of formula (I) with the appropriate acid, for example hydrochloric acid.

### EXAMPLE 1

### N-Demethylation of Oxycodone

Oxycodone (1.19 g) was dissolved in 6 ml DCM and Na₂CO₃ (1.60 g) was added. ACE-Cl (1.56 ml) was added drop-wise to the stirred suspension at room temperature (RT), and the reaction mixture was heated to reflux and stirred for 24 hours. The reaction mixture was filtered and the precipitate was washed with DCM. The filtrate was evaporated to dryness. MeOH (20 ml) was added and the mixture stirred for 1 h at RT. The solution was again evaporated to dryness and added water (25 ml) and conc. HCl (1 ml). The aqueous phase was washed twice with DCM and then added ammonia until pH 11. The aqueous phase was extracted five times with DCM:MeOH mix (80:20). The combined phases from the last extraction was dried and evaporated. Crude noroxycodone was obtained as a white foam (0.73 g, 64%), purity 90% by HPLC.

### EXAMPLE 2

### N-Demethylation of Oxycodone

Oxycodone (0.50 g) and finely powdered Na₂CO₃ (0.67 g) was suspended in DCM (2.5 ml) and ACE-Cl (0.60 ml) was added. The suspension was set to reflux and stirred for 24 hours. The reaction mixture was filtered, concentrated and THF (15 ml) was added together with water (0.50 ml). The solution was stirred at room temperature and a white precipitate started to form. The resulting solid was filtered to yield noroxycodone HCl (0.297 g, 55%), purity 94% by HPLC.

### EXAMPLE 3

### Comparative

In an analogous reaction in which 14-acetoxy oxycodone was used in place of oxycodone, produced complex reaction mixtures from which no noroxycodone could be obtained following hydrolysis.

### EXAMPLE 4

### N-demethylation of Oxycodone Free Base

Oxycodone free base (2.00 g) and finely powdered Na₂CO₃ (6 eq., 4.2 g) was suspended in acetonitrile (10 ml). ACE-Cl (6 eq, 5 ml) was added and the reaction mixture was heated to 50° C. and stirred for 3 days. The inorganic salts where removed by filtration and the solution was concentrated. THF (60 ml) was added together with water (2 ml) and the solution was stirred at 45° C. for 24 hours. The resulting suspension was filtered and the precipitate was dried, yielding noroxycodone hydrochloride (0.970 g, 44% yield) in >95% purity by HPLC.

### EXAMPLE 5

### N-demethylation of Oxycodone to Yield Noroxycodone HCl

To a mixture of oxycodone (58.5 g), sodium carbonate (37.1 g) and TBAB (5.8 g) in acetonitrile (300 ml) in a 1 l reactor kept at 25° C., ACE-Cl (101 ml) was added. The reaction mixture was stirred at 25° C. for 6 hours after which another portion of sodium carbonate (37.1 g) was added. Stirring was continued for 18 hours. The inorganic base was removed by filtration and the filter cake was washed with isopropanol (2×200 ml) and the filtrate was transferred to a 6 l reactor kept at 20° C. Isopropanol (1400 ml) and water (60 ml) was added and the reaction mixture was left stirring at 25° C. for 22 hours and then 23 hours at 0° C. to ensure complete precipitation of the product. The resulting solid was filtered and dried to yield noroxycodone HCl (35.8 g, 57%) as a white solid, 94% pure by HPLC.

### EXAMPLE 6

### N-demethylation of Oxycodone to Yield Noroxycodone HCl

To a mixture of oxycodone (60 g), sodium carbonate (2 eq, 40.3 g) and TBAB (6 g) in acetonitrile (300 ml) in a 1 l reactor kept at 25° C., ACE-Cl (103 ml) was added. The reaction mixture was stirred at 25° C. for 20 hours after which another equivalent sodium carbonate (20.2 g) was added. After another 5.5 hours a fourth equivalent of sodium carbonate (20.2 g) was added and stirring was continued for 4 hours. The inorganic base was removed by filtration and the filter cake was washed with isopropanol (500 ml) and the filtrate was transferred to a 6 l reactor kept at 20° C. Isopropanol (1000 ml) and water (40 ml) was added and the reaction mixture was left stirring at 20° C. for 24 hours to ensure complete precipitation of the product. The resulting solid was filtered and dried to yield noroxycodone HCl (41.7 g, 65%) as a white solid >98% pure by HPLC.

### EXAMPLE 7

### N-demethylation of Oxycodone to Yield Noroxycodone HCl

To a mixture of oxycodone (119 g), sodium carbonate (1 eq, 40.3 g) and TBAB (12 g) in acetonitrile (600 ml) in a 2 l reactor kept at 25° C., ACE-Cl (206 ml) was added. At this point another equivalent of sodium carbonate (40.3 g) was added as well. The reaction mixture was stirred at 25° C. for 18 hours after which another equivalent sodium carbonate (40.3 g) was added. After another 4 hours a fourth equivalent of sodium carbonate (40.3 g) was added and stirring was continued for 3 hours. The inorganic base was removed by filtration and the filter cake was washed with isopropanol (1000 ml) and the filtrate was transferred to a 6 l reactor kept at RT. Isopropanol (2000 ml) and water (96 ml) was added and the reaction mixture was left stirring at RT for 17 hours and at 5° C. for 3 hours to ensure complete precipitation of the product. The resulting solid was filtered and dried to yield noroxycodone HCl (92.3 g, 72%) as a white solid, 98% pure by HPLC.

1. A process for the preparation of a compound of the formula (I) or a pharmaceutically acceptable salt thereof 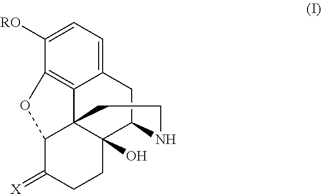
wherein X is CH₂ or O or X is a protected keto group and R is H, CH₃, O.CO.CH₃ or a silyl protecting group, or a salt thereof, which process comprises the reaction of a compound of the formula 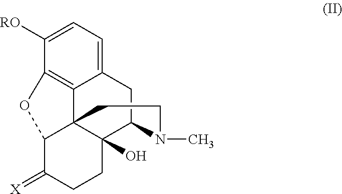
with an α-chloroethyl chloroformate and hydrolysing the resulting intermediate, and optionally forming a pharmaceutical acceptable salt or free base thereof.

2. A process as claimed in claim 1 wherein R is a methyl group.

3. A process as claimed in claim 1 wherein X is O.

4. A process as claimed in claim 1 wherein the intermediate is not isolated prior to hydrolysis.

5. A process as claimed in claim 1 wherein the solvent is acetonitrile.

6. A process as claimed in claim 1 carried out in the presence of a proton acceptor.

7. A process as claimed in claim 6 wherein the proton acceptor is a carbonate or bicarbonate.

8. A process as claimed in claim 1 which commences at and is carried out at an ambient temperature (for example 20-25° C.), or optionally is progressed to a slightly elevated temperature (for example 40-50° C.).

9. A process as claimed in claim 1 wherein the demethylation is carried out in a moisture free environment.

10. A process as claimed in claim 1 wherein the intermediate is hydrolysed with aqueous acid, aqueous THF or aqueous isopropanol.

11. A process as claimed in claim 1, wherein a carbonate or bicarbonate proton acceptor is present.

12. A process as claimed in claim 11, wherein the carbonate proton acceptor is sodium carbonate.

13. A process as claimed in claim 11, wherein the carbonate proton acceptor is added in more than one aliquot, for example, in two or three aliquots.

14. A process as claimed in claim 1 which produces the hydrochloride salt of the compound of formula (I).

15. A process as claimed in claim 1 wherein the demethylation is carried out in the presence of a phase transfer catalyst, for example tetrabutylammonium bromide (TBAB).
